(19) United States
(12) Patent Application Publication
(10) Pub. No.: US 2024/0200004 A1
(43) Pub. Date: Jun. 20, 2024

(54) MULTI-PURPOSE CONTAINER FOR BIOLOGICAL MATERIALS AND METHODS

(71) Applicant: RANAN GÜLHAN AKTRAS, Tuzal, ISTANBUL (TR)

(72) Inventor: Ranan Gülhan AKTAS, Tuzla, Istanbul (TR)

(21) Appl. No.: 18/555,046

(22) PCT Filed: Nov. 2, 2021
(86) PCT No.: PCT/TR2021/051111

§ 371 Date: Oct. 12, 2023

(51) Int. Cl. C12M 1/22; C12M 1/00; C12M 1/04; C12M 1/12

(52) U.S. Cl. CPC C12M 23/10; C12M 23/24; C12M 23/38; C12M 25/14; C12M 29/04

(57) ABSTRACT

Disclosed is a multi-purpose container that provides a single safe environment for multiple processes during two-dimensional and three-dimensional culture of biological materials. This container is developed to protect the quantity and quality of the material in one place during different processes. These processes may include culturing, freezing, thawing, staining, immunolabeling, examining under optical microscopes (e.g., light, fluorescence, confocal microscopes), preparing samples for histological or electron microscopic examination or molecular assays, and making ready the specimen for transplantation or implantation. In one example, container has two parts. The lower part contains central portion for holding the biological material. The upper portion may include openings to allow gas flow. There might also be an alternative central portion of the upper part. The closing system between the upper and lower parts provides two positions, one allowing a gas flow and one blocking the gas flow.

### TECHNICAL FIELD

The present invention relates to a container designed to create a single safe environment for multiple processes of biological materials. These processes preferably comprise two-dimensional or three-dimensional culturing, co-culturing, staining, immunolabeling, freezing, thawing, examining under optical microscopes-like brightfield, darkfield, fluorescence, confocal, and super-resolution microscopes-, preparing samples for histological analysis, molecular assays, electron microscopy, transplantation, or implantation.

Two-dimensional and three-dimensional culture technologies are great tools for pre-clinical, translational, and clinical studies and include various methodologies like microscopy, molecular assays, freezing, transplanting, or implanting the biological material. Using these methods requires transferring the material between various culture vessels, including flasks, cell culture dishes, multi-well plates, strainers, centrifuge tubes, Eppendorf tubes, and cryo-tubes. Centrifugation and transfer of sensitive and fragile biological materials from one container to another during those processes cause significant damage and loss of the precious material. A single container that is available for several sequential processes will maximize the viability, decrease the chance of the loss of the unique material, minimize the cost and labor need, save time, and reduce difficulties related to handling sensitive materials.

### BACKGROUND

Two-dimensional and three-dimensional culture techniques are used for processing different biological materials (e.g., cells, cell lines, organoids, spheroids, embryos, microorganisms, and other living organisms) to address biological and pathological questions. Two-dimensional cell culture methods have been in use since the early 1900s. They involve growing cells on a flat surface, such as the bottom of a petri dish or a flask. Three-dimensional cell culture techniques are artificially created environments in which biological materials are grown in three dimensions. Those three-dimensional structures are grown in a substrate that might be an artificial matrix or a scaffold and mimic the native environment. The hanging drop technique is the first three-dimensional culture method that Dr. Ross Granville developed. Recently, organoids and spheroids have become popular three-dimensional models. Those techniques have brought new capabilities and better performance to the lab. They mimic the “in-vivo” world and hold remarkable promise for disease modeling, personalized medicine with the potential to help study tissue repair, developmental biology, and drug responses.

However, there are challenges in the field since the quality control measures seem necessary to improve the reliability and reproducibility of data. In addition, those biological materials contain highly fragile and sensitive structures like stem cells, neurons, organoids, and spheroids. The current approaches have limitations in preserving the materials during the whole procedure since those biological materials are being centrifuged, harvested, and transferred between different containers during those processes.

For example, the sample is transferred from a cell culture dish, plate, or flask to a tube for centrifugation and then moved to cryo-containers (e.g., cryotubes, straws, bags) for freezing. For thawing the same sample, the material is again carried into a new tube, centrifuged, and then transferred into another container-like culture dish, flask, or a well plate. For confocal microscopic and electron microscopic examinations, the material is moved to a tube, centrifuged, processed, and immunolabeled with specific antibodies in different containers until the material is ready to be replaced on the glass surface for confocal microscopic examination or on the grid for electron microscopic investigation. Handling, centrifuging, and carriage the biological material through those sequential steps cause additional harmful adverse effects and deformation, reduce viability, and significantly diminish the amounts of viable materials. In addition to that, trypsinization of the cells to detach from the culture surface causes changes in the quality of the sample.

Furthermore, those steps seriously damage the three-dimensional structures (e.g., organoids, spheroids, embryos) and the cells due to the lengthy processing (e.g., sperms, neurons, stellate cells). Additionally, there can be a wide variation in experimental results due to variation in actions in different culture vessels. Another example is the conventional methods for subculturing and/or examining the cells by using molecular assays. Every time the cells need to be examined, the steps of detaching the cells from the culture container, diluting the cells, and centrifuging, seeding, subculturing, or processing for an assay in a new culture container are repeated. These steps cause changes in the cell phenotype and proliferation capacity.

Transplantation and implantation of biological materials-like organoids- to the living organism is becoming one of the most valuable pre-clinical tools in research. However, the material's quality and quantity after sequential processes are the current protocols' main limitations. Poor quality of the processed material is the fundamental reason for the failure of in vivo growth of these materials. Similarly, cell transplantation to the patients has been placed between the fascinating research area in medicine. It opens new doors for translational studies and regenerative medicine. Current preparation techniques of the cells in in-vitro conditions require subsequent steps with carrying the developing materials from one environment to another. A container that allows the cells or any other biological material to stay in a single place from the beginning until their transplantation or implantation would be beneficial since it will optimize in vitro sampling. A single container for all steps will preserve the quality and quantity of the biological material since the steps described above will be omitted. Again, it will reduce labor time and errors associated with passaging, centrifuging, transporting the structures to other media.

Taken together, those methods based on trypsinization, centrifugation, and transfer of the biological material between different containers cause difficulty in standardizing and integrating the conclusions. The sequential steps described above result in losing or damaging the biological materials. During those procedures, proliferative capacity decreases, and cells degraded in such a way may affect the test results. As a result, outcomes at the end may not reflect the conditions of the biological materials at the beginning of the process. Because of those multiple steps in different containers, the experiments' consistency, efficiency, and reproducibility are still a big problem using current methodologies. Researchers try to find out how to enhance their research capabilities through safer culture devices, uniquely designed vehicles, smart controls in incubation, freezing, thawing, microscopic examination, and transplantation while preventing contamination.

Therefore, it is desirable to improve the current conventional technologies to have biological materials with stable quality. A method/device that will skip the centrifugation and several other steps and provide the material in a single safe environment from the beginning to the end of the multiple processes would be beneficial to reach the most consistent and reliable data. Additionally, this will be time, labor, and cost-saving.

Glass-bottomed dishes and multichannel slides are commercialized products that provide a single environment for the cells to be cultured and then examined under a fluorescence or confocal microscope. The glass portion of those culture vessels provides a transparent surface with no autofluorescence. The cells are cultured, labeled, and examined under a microscope by using those two different vessels. However, the users who plan to freeze the cells or perform other methods like electron microscopy or molecular assays cannot use those containers. Additionally, those vessels are not suitable for co-culture studies and preparation of the samples for transplantation or implantation.

No current commercial device allows processing the biological materials from the beginning to the end of the various processes, including culturing, co-culturing, freezing, thawing, immunostaining, examining under microscopes, preparing the biological materials for electron microscopy, transplantation or implantation.

Additionally, there is no current solution to secure an experiment if there is a sudden interruption of the studies due to a change of circumstances. A device that allows the researcher to stop the investigation in an urgent situation by freezing them in their current situation and then continue without any loss when the conditions are favorable would be highly beneficial; since it will save time, labor, and cost. For example, because of the COVID pandemic, scientists needed to stop the experiments immediately and lose their precious materials and data. Another situation occurs when there is a need for a specialized device or reagent to halt the investigation and freeze the specimen until a new reagent or equipment arrives at the lab. There is no current solution to hold the sample in a safe environment for long-term until that device or reagent comes to the lab.

Accordingly, the present applicant has recognized the need for an improved device for performing the procedures described above in a single multi-purpose container, eliminating steps like centrifuging, and transferring the material to multiple culture containers.

The container and the methods described here may provide advantages over conventional culture devices since the material will be in the same environment from the beginning to the end without centrifuging and carriage to other culture vessels. The structure of the biological material will be protected since the biological material will remain in the same place during multiple processes. The present invention relates both to two- and three-dimensional culture platforms for culturing, freezing, thawing, analyzing biological materials under different microscopes-like brightfield, darkfield, fluorescence, confocal, and super-resolution microscopes-, preparing them for further analytical methods-like histology, electron microscopy, and molecular assays-, and preparing the material for transplantation or implantation. The container minimizes the loss of information related to the biological materials' structure, providing consistency, and ensuring the experiment's performance. These and other advantages of the various embodiments of the containers described herein will be readily apparent to those of skill in the art upon reading the disclosure presented herein.

### BRIEF SUMMARY OF THE INVENTION

The current invention, the container, provides an environment for biological materials to perform subsequent procedures in one device—from the beginning of culturing to examining or preparing for various processes, including transplantation or implantation. The materials stay in a single container during different processes and do not move from one container to another, the quantity of the material does not change, and the structure of the biological material remains intact. Furthermore, this container minimizes the loss of information and retains the high viability and quality of the material since the methods with this container exclude centrifuging and other harmful steps due to the transfer of the material between different vehicles and minimize handling difficulties.

In order to achieve the aims of the invention, a multi-purpose container comprising:
a lower part with a central portion with a raised rim around the central portion,
an upper part that might contain a central portion,
a closing system that provides two positions, one sealed and the other allowing gas flow,
   has been improved.

The invention provides a safe and single environment during different processes for the biological materials. These processes comprise but are not limited to: 1. Two-dimensional and three-dimensional culturing, 2. Freezing and thawing, 3. Histological staining, 4. Immunolabeling, 5. Examining under various optical microscopes (e.g., darkfield, brightfield, fluorescence, confocal, super-resolution), 6. Preparing the biological material for histology, electron microscopy, molecular assays, transplantation or implantation. In different embodiments of said container, the upper parts and/or the lower parts and/or the central and peripheral portions are made of different biocompatible and/or bioresorbable materials and/or composed of various shapes and/or include compartments, chambers, additional portions in compliance with the multiple processes described herein.

In one embodiment of said container, the central portion and/or the peripheral portion of the lower and/or the upper part are made of the following materials in compliance with the processes:
a. Glass
b. Chlorotrifluoroethylene—also called ACLAR33C film—
b. Polyvinylchloride
c. Polyethersulfone
d. Polytetrafluoroethylene
e. Polyethylene
f. Polyurethane
g. Polyetherimide
h. Polycarbonate
i. Polysulfone
j. Polyetheretherketone
k. Polypropylene
l. Polystyrene
m. Fluoropolymer
n. Any other polymer
o. Any other biocompatible material
p. Any other bioresorbable material

In one embodiment of said container, the upper part contains pores for gas flow.

In one embodiment of said container, the upper part contains a filter inside to minimize contamination.

In some embodiments, both the lower and upper parts of the containers include central portions to allow the user to use both parts' features for experiments and apply hanging drop methodology and multiple processes.

In one embodiment of said container, the invention includes a central portion with concave or conical bottom, which allows the biological material to settle in a central location away from the niche walls and helps the user efficiently handle the limited number of biological materials. In addition, the embodiment offers a better orientation for optics and gives the advantage of group culture and multiple processes of the material.

In one embodiment of said container, the lower central portion and/or the upper central portion contain multiple compartments for different materials or different experiments.

In one embodiment of said container, the upper and/or the lower central portions contain movable portions that are made of the following materials:
a. Glass
b. Chlorotrifluoroethylene—also called ACLAR33C film—
c. Polyvinylchloride
d. Polyethersulfone
e. Polytetrafluoroethylene
f. Polyethylene
g. Polyurethane
h. Polyetherimide
i. Polycarbonate
j. Polysulfone
k. Polyetheretherketone
l. Polypropylene
m. Polystyrene
n. Fluoropolymer
o. Any other polymer
p. Any other biocompatible material
q. Any other bioresorbable material
r. Filtered membrane containing pores with a specific size for the biological material
s. Grid for transmission electron microscopy

In one embodiment of said container, a movable portion contains a filtered membrane. In this embodiment, the three-dimensionally growing cells, organoids, spheroids, and other living organisms in a gel-like Matrigel, hydrogel, and others may be cooled to liquefy the surrounding extracellular matrix and then filtered for further molecular assays without centrifuging.

In one embodiment of said container, the biological material is grown on the grid, examined under various microscopes, and then processed in the same movable portion for transmission electron microscopy.

In one embodiment of said container, the lower part and/or the lower movable central portion has a separate cap that is made of the following materials:
a. Glass
b. Chlorotrifluoroethylene—also called ACLAR33C film—
c. Polyvinylchloride
d. Polyethersulfone
e. Polytetrafluoroethylene
f. Polyethylene
g. Polyurethane
h. Polyetherimide
i. Polycarbonate.
j. Polysulfone
k. Polyetheretherketone
l. Polypropylene
m. Polystyrene
n. Fluoropolymer
o. Any other polymer
p. Any other biocompatible material
q. Any other bioresorbable material
r. Filtered membranes containing pores with a specific size for the biological material
s. Grid for transmission electron microscopy

Another embodiment of this invention is a container containing structural parts, which are covered or filled with components to promote adhesion, three-dimensional growth, and/or differentiation of the biological material during various processes. The upper and/or lower central parts are covered and/or filled and/or made off with any of the following reagents:
a. Collagen
b. Matrigel
c. Laminin
b. Fibrinogen
c. Matrigel
d. Hydrogel
e. Alginate
f. Fluoropolymer
g. Any polymer for transplantation/implantation
v. Synthetic or natural any other extracellular matrix components
v. Any other biocompatible materials including various extracellular matrix components

In one embodiment of said container, the upper and the lower central parts contain scaffolds.

In one embodiment of said container, the container is designed at different sizes and/or shapes and/or contain multiple niches.

In one embodiment of said container, the container is designed to provide specific desired shapes and sizes for the culture of the cells needing ultra-low attached surfaces or shaped for adherent surfaces.

In one embodiment of said container, the lower and/or upper parts have chambers and/or layers connected with channels to provide an environment for co-culture experiments.

Another embodiment of this invention is the inclusion of holes on the sides of the upper and lower parts to connect to external microfluidic devices.

In one embodiment of said container, the container is embedded in an additional thermal insulator part made of the following: Aerogel, fiberglass, cellulose, polystyrene, and others. That embodiment allows lengthening the processes in certain conditions—like when the sample is needed to be held in liquified Matrigel for an extended period.

In all embodiments of the present invention, the culture media, or other appropriate chemicals (e.g., freezing, or thawing solutions) are administered locally to the central portion and/or to the chambers and/or to the compartments and/or to the niches.

The methods for the following procedures are also disclosed: 1. Culture of biological material, 2. Freezing and thawing, 3. Hanging-drop methodology, 4. Molecular assays, 5. Immunocytochemistry and immunohistochemistry, 6. Electron microscopy, 7. Paraffin-embedded sample preparation 8. Visualization of the biological material under fluorescence, confocal or super-resolution microscope.

### BRIEF DESCRIPTION OF THE DRAWINGS

Certain illustrative examples are described below with reference to the accompanying figures in which:

FIG. 1A illustrates an example of a plan view of lower and upper parts of a container in accordance with certain examples;

FIG. 1B illustrates an example of a cross-sectional view of the lower part of a container in accordance with certain examples;

FIG. 1C illustrates an example of a plan view of the lower part of a container in accordance with certain examples;

FIG. 1D illustrates an example of a side view of the lower part of a container in accordance with certain examples;

FIG. 1E illustrates an example of an interior plan view of the upper part of a container in accordance with certain examples;

FIG. 1F illustrates an example of an oblique cross-sectional view of the upper part of a container in accordance with certain examples;

FIG. 1G illustrates an example of an interior cross-sectional view of the upper part of a container in accordance with certain examples;

FIG. 1H illustrates an example of a side view of the upper part of a container in accordance with certain examples;

FIG. 2A illustrates an example of an oblique cross-sectional view of the upper part of a container in accordance with certain examples;

FIG. 2B illustrates an example of an oblique view of the upper part of a container in accordance with certain examples;

FIG. 2C illustrates an example of an exterior plan view of the upper part of a container in accordance with certain examples;

FIG. 3A illustrates an example of a cross-sectional interior view of the upper part of a container in accordance with certain examples;

FIG. 3B illustrates an example of an interior view of the upper part of a container in accordance with certain examples;

FIG. 3C illustrates an example of an interior oblique view of the upper part of a container in accordance with certain examples;

FIG. 4A illustrates an example of an interior view of the lower part of a container in accordance with certain examples;

FIG. 4B illustrates an example of an oblique view of the lower part of a container in accordance with certain examples;

FIG. 4C illustrates an example of a cross-sectional view of the lower part of a container in accordance with certain examples;

FIG. 4D illustrates an example of a side view of the lower part of a container in accordance with certain examples;

FIG. 5A illustrates an example of an interior view of the lower part of a container in accordance with certain examples;

FIG. 5B illustrates an example of an oblique view of the lower part of a container in accordance with certain examples;

FIG. 5C illustrates an example of a cross-sectional view of the lower part of a container in accordance with certain examples;

FIG. 5D illustrates an example of a side view of the lower part of a container in accordance with certain examples;

FIG. 6A illustrates an example of an interior view of the lower part of a container in accordance with certain examples;

FIG. 6B illustrates an example of an oblique view of the lower part of a container in accordance with certain examples;

FIG. 6C illustrates an example of a cross-sectional view of the lower part of a container in accordance with certain examples;

FIG. 6D illustrates an example of a side view of the lower part of a container in accordance with certain examples;

FIG. 7A illustrates an example of an interior view of the lower part of a container in accordance with certain examples;

FIG. 7B illustrates an example of an oblique view of the lower part of a container in accordance with certain examples;

FIG. 7C illustrates an example of a cross-sectional view of the lower part of a container in accordance with certain examples;

FIG. 7D illustrates an example of an oblique cross-sectional view of the lower part of a container in accordance with certain examples;

FIG. 7E illustrates an example of an oblique cross-sectional view of the lower part of a container when the central portion is moved in accordance with certain examples;

FIG. 8A illustrates an example of a cross-sectional view of the lower part of a container in accordance with certain examples;

FIG. 8B illustrates an example of an oblique cross-sectional interior view of the lower part of a container in accordance with certain examples;

FIG. 8C illustrates an example of an oblique cross-sectional interior view of the lower part of a container, when the cap is moved, in accordance with certain examples;

FIG. 9A illustrates an example of an oblique interior cross-sectional view of the lower part of a container, when the part containing chambers and channels is moved, in accordance with certain examples;

FIG. 9B illustrates an example of an oblique cross-sectional view of the lower part of a container in accordance with certain examples;

FIG. 9C illustrates an example of a cross-sectional view of the lower part of a container in accordance with certain examples;

FIG. 9D illustrates an example of an interior view of the lower part of a container in accordance with certain examples;

FIG. 10A illustrates an example of an oblique cross-sectional interior view of a closed container in accordance with certain examples;

FIG. 10B illustrates an example of plan views of the lower and upper parts of an open container in accordance with certain examples;

FIG. 10C illustrates an example of a plan view of a closed container in accordance with certain examples;

FIG. 11A illustrates an example of a cross-sectional view of a closed container in accordance with certain examples;

FIG. 11B illustrates an example of a side view of a closed container in accordance with certain examples.

### DESCRIPTION OF REFERENCES

1. Container
11. Lower part
12. Upper part
13. Lower part peripheral portion
14. Lower part central portion
15. Lower part rim
16. Screwed closing system
17. Upper part central portion
18. Upper part peripheral portion
21. Pores
32. Upper part rim
41. Central portion with conical bottom
51. Central portion with concave bottom
61. Central portion with multiple compartments
71. Central movable portion
81. Cap
91. Chamber
92. Layer
93. Channel
101. Holes
111. Thermal insulator portion

### DETAILED DESCRIPTION OF THE INVENTION

The examples and embodiments described below are primarily in connection with a container (1), such as a culture dish, suitable for a range of procedures and processes during and after two-dimensional and three-dimensional cultures of biological materials. Those biological materials comprise but are not limited to cells, cell lines, organoids, spheroids, embryos, microorganisms, tissue samples, biopsy specimens, and other living organisms. The procedures and processes comprise but are not limited to culturing, freezing, thawing, histological analysis, immunostaining, examining under a variety type of microscopes, preparing samples for electron microscopy, getting ready the cells for molecular assays, co-culturing, preparing the samples for transplantation or implantation, and attaching the container (1) to a microfluidic system. The described container (1) may also be used in connection with a range of medical procedures and methods in various environments and temperatures.

In the following detailed description, reference is made to the accompanying drawings that form a part hereof, and in which are shown by way of illustration several specific embodiments of containers (1), systems, and methods. It is to be understood that other embodiments are contemplated and may be made without departing from the scope or spirit of the present disclosure. The following detailed description, therefore, is not to be taken in a limiting sense. All scientific and technical terms used herein have meanings commonly used in the art unless otherwise specified. The definitions provided herein facilitate understanding specific terms frequently used herein and are not meant to limit the scope of the present disclosure. As used in this specification and the appended claims, the singular forms “a,” “an”, and “the” encompass embodiments having plural referents, unless the content dictates otherwise.

As used in this specification and the appended claims, the term ‘or’ is generally employed in its sense, including “and/or unless the content dictates otherwise.

As used herein, “have,” “having,” “include,” “including, “comprise,” “comprising,” or the like are used in their open-ended sense and generally mean “including, but not limited to.”

Any direction referred to herein, such as “bottom,” “upper,” “lower,” “above,” below,” “top”, and other directions and orientations are described herein for clarity in reference to the figures and are not to be limiting of an actual container (1) or system or use of the container (1) or system. Containers (1) or methods as described herein may be used in a number of directions and orientations.

As used herein, “central portion”, “compartment”, “chamber” and/or “niche” are located in both the lower and upper parts (12) of the container (1) for placing, culturing, and processing the biological material. The material, size, and shape of the “central portion”, “compartment”, “chamber”, and/or “niche” differ in different embodiments.

As used herein, “biological material” means cells, organoids, spheroids, tissue samples, stem cells, primary cells, cell lines, embryos, oocytes, sperms, microorganisms, biopsy specimens, and other living organisms; but are not limited to.

As used herein, “closing system” means a mechanism with screws or clips and a gasket that provides two positions, one sealed and the other allowing gas flow.

Certain embodiments of the present invention can be used for the following purposes but are not limited to;
a. Culturing of biological materials (including but not limited to cells, organoids, spheroids, tissue samples, stem cells, primary cells, cell lines, embryos, oocytes, sperms, tissue samples, biopsy specimens, living organisms)
b. Microscopic analysis of the biological materials
c. Freezing and thawing the biological materials
d. Co-culturing of the biological materials
e. Hanging drop methodology usage for biological materials
f. Preparing the biological materials for electron microscopic analysis
g. Preparing the biological materials for molecular assays
h. Preparing the biological material for routine histological analysis and staining
i. Preparing the biological material for transplantation or implantation
j. Providing a single environment for the biological materials during the whole process when it is attached to a microfluidic device

Certain embodiments allow the user to culturing, freezing, thawing, culturing again and then processing in the same container respectively (1).

In certain embodiments, the container (1) is partially or totally made of copolymer film consisting primarily of Chlorotrifluoroethylene (CFTE)—also called ACLAR33C. It is a good choice for growing biological materials since it is chemically stable and biochemically inert. Additionally, it is crystal clear and provides high UV transparency, making it ideal for UV curing to embed resin in microscopy. Since it exhibits no autofluorescence, it is suitable for fluorescence and confocal microscopy in addition to brightfield imaging. It is flexible and soft and can be sectioned without damage to the ultramicrotome knives. Low dielectric constant, high electric strength and low dissipation factor offer excellent attachment even through lengthy processing procedures. It provides a great moisture barrier, excellent chemical resistance, and minimal dimensional change (less than 2%), making it an ideal choice for immunostaining experiments in addition to microscopy. Because of low surface energy, it is easy to separate from epoxy and extracellular matrix-like Matrigel. It can be sterilized by heat or UV. Taken together, Chlorotrifluoroethylene is a good candidate for the various processes of the biological material from the beginning to the end. For example, the biological material can be cultured, immunolabeled, examined under fluorescent microscopes, and/or prepared for electron microscopic examination on this plastic. It is also ideal for freezing and thawing since it is resistant to temperatures between −160° C. and +200° C.

In certain embodiments, the central portion of the lower part (14) and central portion of the upper part (17) are made of transparent biocompatible materials to provide image analysis under different microscopes during multiple processes.

In certain embodiments, the container (1) includes a movable part (71). Either central portion (14) or this movable part (71) can be made of different biomaterials according to the container's (1) purpose. For example, either central portion (14) or movable parts (71) might be covered or filled with extracellular matrix components, hydrogels, scaffolds, and/or others. The movable part (71) is made of Chlorotrifluoroethylene to prepare the sample for histological investigation and electron microscopy and examine the material under various microscopes after culturing. The movable part (71) with specific biocompatible and bioresorbable material is used to transplant or implant the biological material. For example, it is made of any of the following materials for following particular purposes but not limited to: 1. Hydroxypropyl methylcellulose phthalate for preparation of enteric-coated samples 2. Polyvinylchloride for the 3D culture of blood cells, 3. Polyethersulfone for transplantation of 3D growing cells that will be loaded to catheters, 4. Polytetrafluoroethylene for tubing, synthetic blood vessels, surgical sutures, reconstructive surgery, and soft tissue regeneration patches, 5. Polyethylene for surgical cables, artificial tendons, and orthopedic sutures, tubing, 6. Polyurethane for wound applications, 7. Polyetherimide for skin applications, 8. Polycarbonate when it is needed glasslike transparency, 9. Polysulfone for surgical and medical containers, or artificial heart components, heart valves, 10. Polyetheretherketone for dentistry products and rigid tubing, 11. Polypropylene for heart valves.

In certain embodiments, the lower central portion (14) and/or the upper central portion (17) and/or the movable portion (71) contain secure caps (81). That allows protection of the biological material in certain conditions during multiple processes.

In certain embodiments, the user can perform co-culture experiments since the embodiment includes chambers (91), layers (92), and channels (93). These embodiments provide a protective environment for the biological materials that have been co-cultured and processed for multiple purposes.

In another embodiment, holes (101) located on the sides of the container (1) provide a connection between the container (1) and a microfluidic system while performing subsequent processes.

Throughout this specification, unless the context requires otherwise, the words ‘comprise” and “include” and variations such as “comprising” and “including” will be understood to imply the inclusion of an item or group of items, but not the exclusion of any other item or group items.

Although the invention herein has been described with reference to embodiments, it is to be understood that these embodiments are merely illustrative of the principles and applications of the present invention. Therefore, it is to be understood that these and various other omissions, additions, and numerous modifications may be made to the illustrative embodiments. The different arrangements may be devised without departing from the spirit and scope of the invention defined by the appended claims.

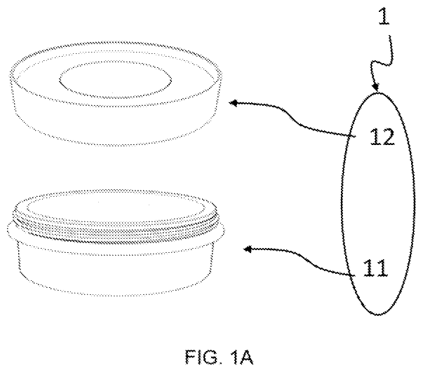

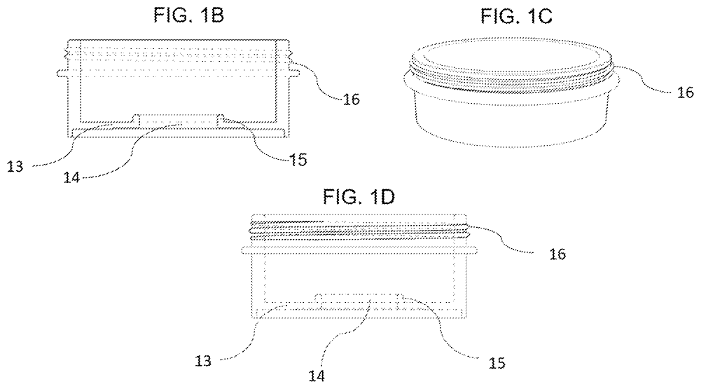

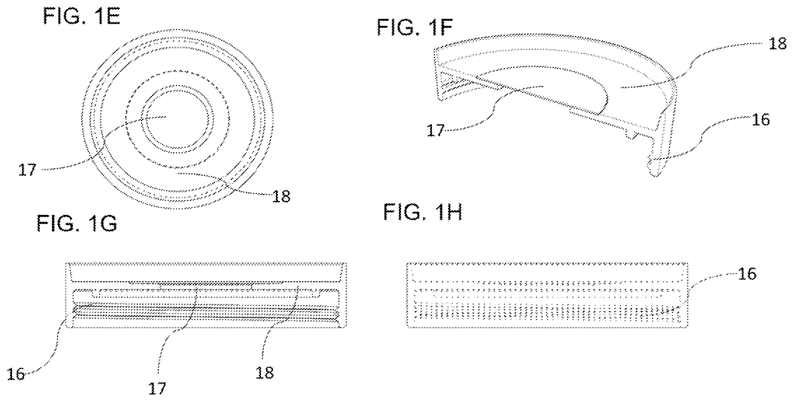

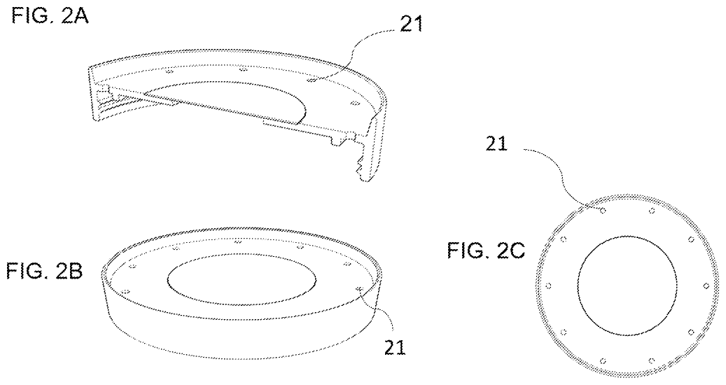

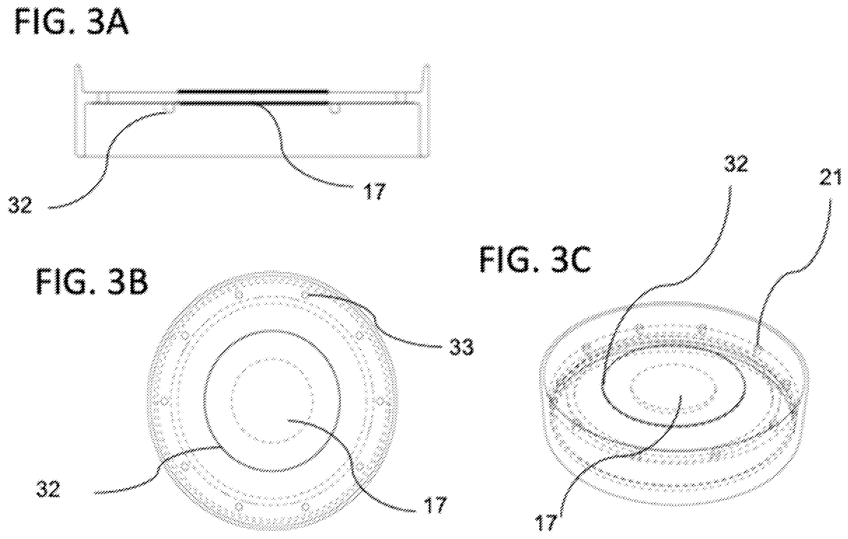

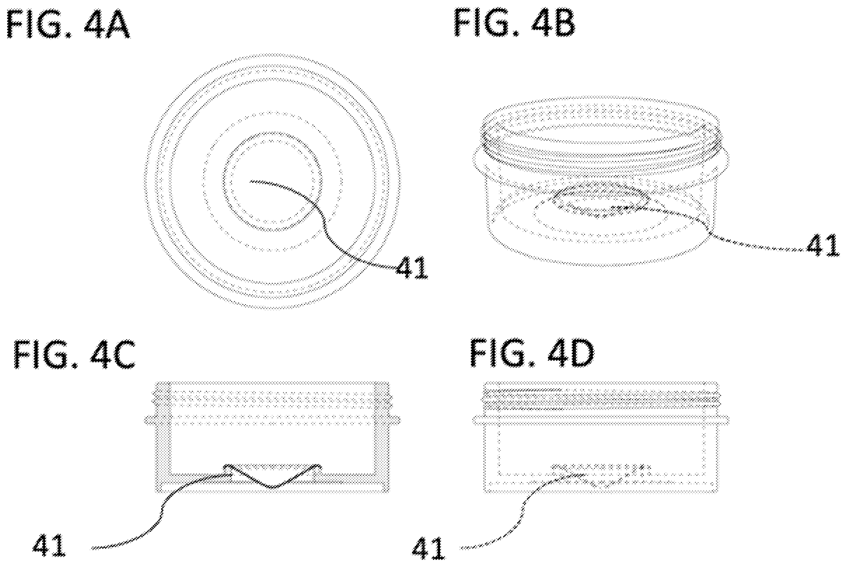

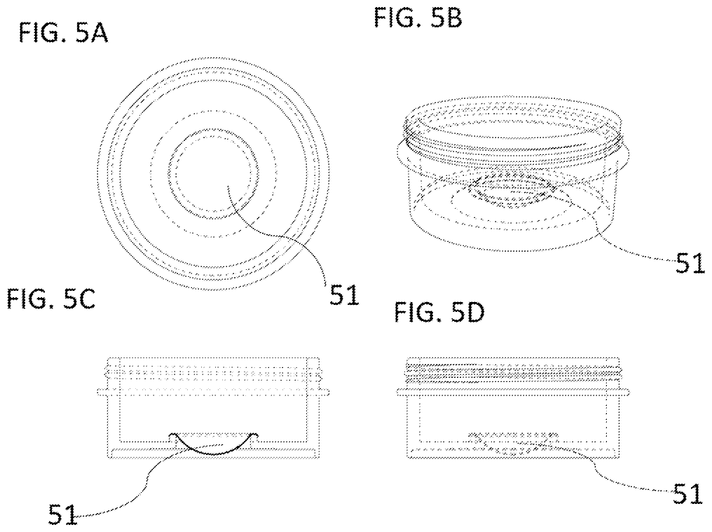

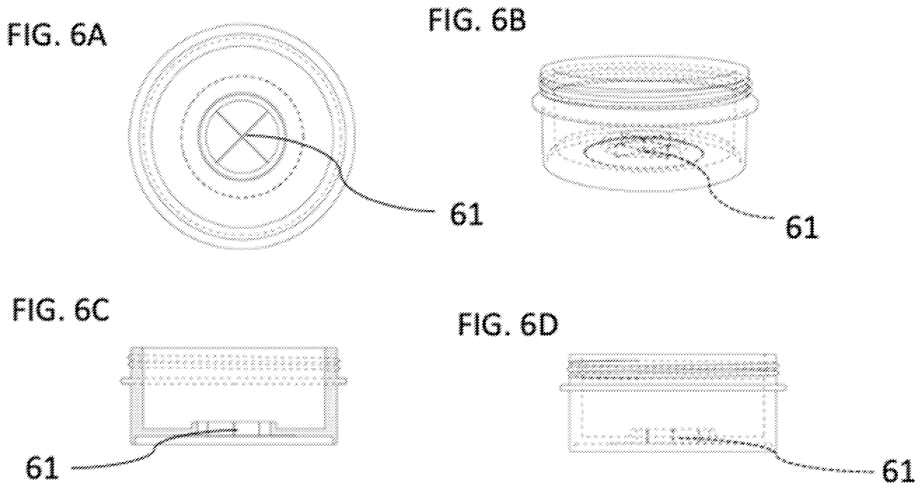

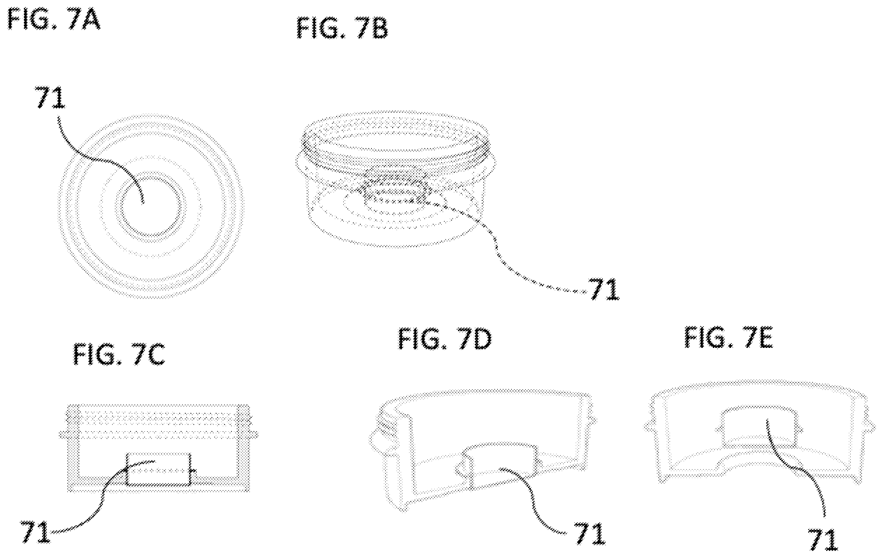

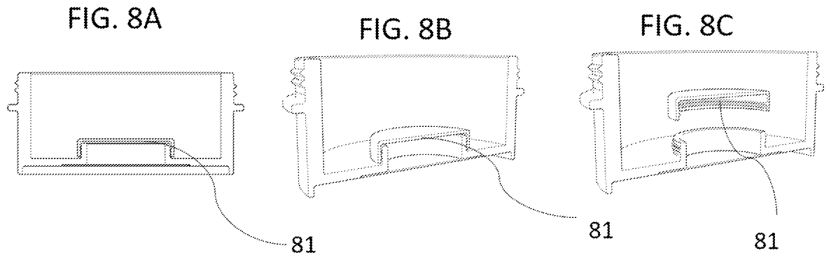

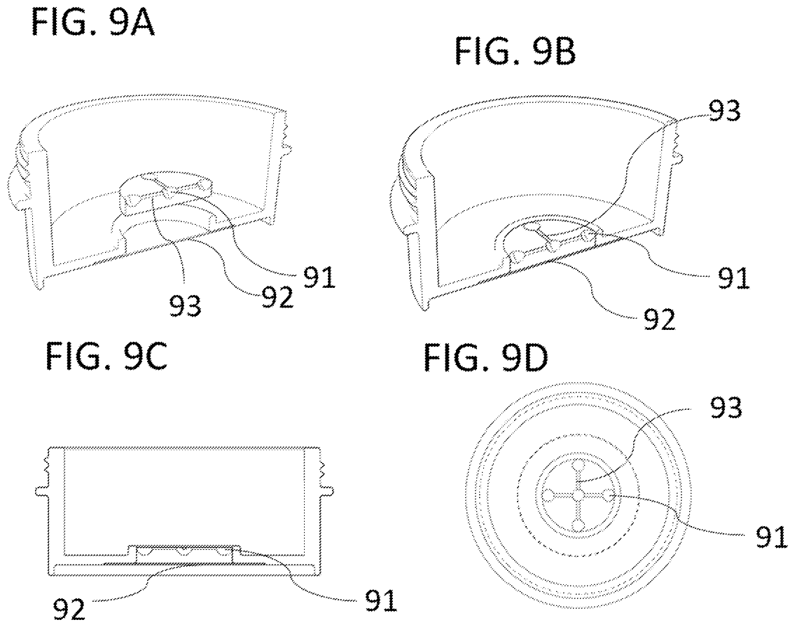

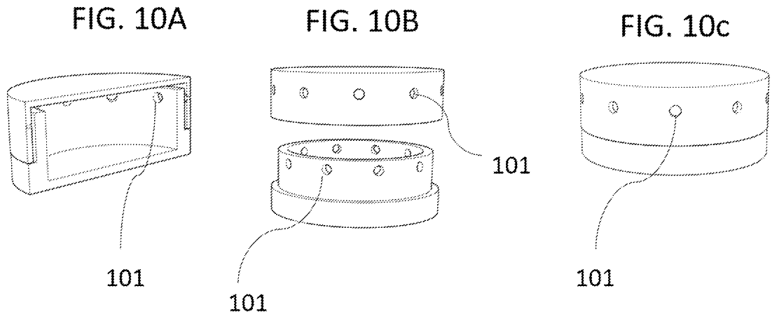

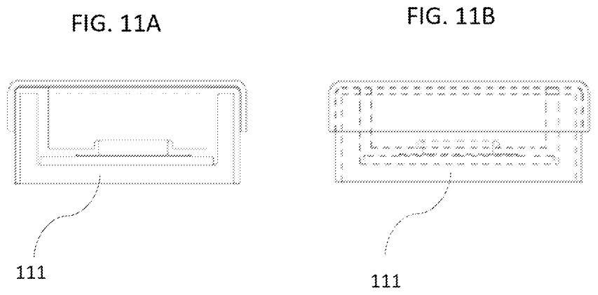

Referring now to figures, in one embodiment, FIG. 1A illustrates the upper and the lower parts with the central portions and closing system with screws. The lower part central portion (14), the upper part central portion (17), compartments (61), the chambers (91), and the niches are the locations for biological materials to be cultured and processed FIGS. 1B-D displays details of the lower part and FIGS. 1E-H illustrates the upper part of one embodiment. As illustrated in FIGS. 2A, B, and C; pores (21) on the upper part (12) of one embodiment allow the gas flow. FIGS. 3A, B, and C are related to an embodiment with an upper part (12) containing a central portion (91) and pores (21). FIGS. 4A-D and FIGS. 5A-D are examples demonstrating various shapes of the central portion; one is conical while the other is concave for specific purposes. FIGS. 6A-D illustrates the lower part of an embodiment with multiple compartments. As in FIGS. 7A-E, there is a movable central portion (71) in one embodiment. FIGS. 8A-C illustrates a central portion (14) with a cap (81) to protect the biological material for various purposes. According to FIGS. 9A-D, chambers (91), layer (92) and channels (93) provide co-culture conditions for biological material and create an organ-on-a-chip device to perform multiple processes described herein. According to FIGS. 10A-C, lower and upper portions of one embodiment contain holes (101) on the sides of the lower and upper parts to provide the connection with a microfluidic system during various processes. As in FIGS. 11A and B, a thermal insulator portion (111) serves as a heatproof for the biological material.

The upper part (12) of different embodiments might contain central portions of different sizes and shapes with other materials that have been described above for the lower parts (11) of the container (1). The container (1) might contain more than one compartment/chamber/central portion in both the lower part (11) and the upper part (12).

The following methods are used for processing the biological material in the container (1) according to the invention, but are not limited to any of the methods described here:

A. The user can use both the upper part (12) and lower part (11) of the container (1) for two-dimensional and three-dimensional culture techniques and choose the most suitable embodiment for the subsequent multiple processes for each experiment.

B. For freezing, either the whole container (1) or the movable part with or without the cap (81) in the container is used. Conventional cryopreservation methods, slow freezing, and/or the vitrification method are used to freeze and thaw the biological material in the same container (1) with the surrounding environment, like the extracellular matrix. Slow freezing or vitrification methods with compatible freezing agents for the biological material are preferred for sensitive materials (e.g., oocytes, sperms, embryos, neurons, organoids, spheroids). If there is a gel-like extracellular matrix surrounding the biological material, the first freezing steps should be carried out at 37° C. to prevent liquefication of the gel. The user should ensure that the container (1) or the central portion with the cap (81) is firmly closed for freezing. The method further comprises transferring the container (1) or the movable part with the cap into the storage device (e.g., −20° C. freezer, −80° C. freezer, nitrogen storage tanks). The thawing agents and methods to keep the biological material in the container (1) without moving during subsequent processes depend on the biological material's nature. For example, vitrification solutions are recommended for freezing and thawing the sperms and the oocytes.

C. For hanging drop methodology, the lower part (11) and upper part (12) of the container (1) can be used for culturing while using the container (1) up-side-down and regularly changing the lower part (11) and the upper part (12).

D. For molecular biology assays after culturing, the container (1) that has a filter in the movable part is used. First, container (1) is left on the ice to let the extracellular matrix liquify, and the filter allows the biological material to stay at the top. This step is used to isolate the biological material to the surrounding extracellular matrix, and no centrifugation is needed. Then, the following planned processes are applied to the material.

E. For immunohistochemistry and immunocytochemistry experiments and culturing of the material, the container (1) with the central part made of glass, Chlorotrifluoroethylene (CFTE—also called ACLAR33C) film or any available transparent plastic is used. The following two methods might be applied but are not limited to:
(i) If it is two-dimensional cell culture experiment, common immunostaining methods are applied at room temperature, or a fast method is applied at 37° C.
(ii) If a three-dimensional cell culture experiment is performed and an extracellular matrix surrounds the biological material, all immunolabeling steps are carried at 37° C.

F. For electron microscopy of the biological material, two methodologies with two different embodiments are available: 1. The biological material is grown on the movable portion (71) composed of a grid and then processed (e.g., labeled and stained) in the same movable portion for transmission electron microscopy. 2. The container (1) with chlorotrifluoroethylene—also called ACLAR33C film—is used for culturing and then conventional embedding steps for electron microscopy in the container (1) without moving the biological material. If an extracellular matrix surrounds the biological material, the actions are carried at 37° C. After embedding into the resin, an ultramicrotome knife can section the material surrounded by the film. It is also possible to separate the resin block from chlorotrifluoroethylene film and continue sectioning.

G. For the preparation of paraffin-embedded block after culturing the biological material, the Chlorotrifluoroethylene—also called ACLAR33C film- or any other biocompatible plastic is used. The conventional embedding steps are applied in container (1) without moving the biological material. All actions are carried at 37° C. until embedding into paraffin if an extracellular matrix surrounds the biological material. After preparation of paraffin embedded sample, sectioning and staining might be applied.

H. To visualize the biological material under fluorescence, confocal or super-resolution microscope, the lower central portion (14) and/or the upper central portion (17) should contain proper transparent material for optic analysis, like glass, Chlorotrifluoroethylene (CFTE—also called ACLAR33C) film, or another type of specific, translucent plastic. The user can culture. Label, and directly examine the material after inserting the container (1) into the place under the microscope.

1. A multi-purpose container comprising:
a lower part with a lower part central portion with a raised lower part rim around the lower part central portion;
an upper part; and
a closing system that provides two positions, one sealed and the other allowing gas flow.

2. A container according to claim 1, characterized in that wherein the upper part has an upper part central portion, wherein the lower part central portion and/or upper part central portion and/or lower part peripheral portion and/or upper part peripheral portion which are made of from one of the following materials:
a. Glass
b. Chlorotrifluoroethylene—also called ACLAR33C film—
b. Polyvinylchloride
c. Polyethersulfone
d. Polytetrafluoroethylene
e. Polyethylene
f. Polyurethane
g. Polyetherimide
h. Polycarbonate
i. Polysulfone
j. Polyetheretherketone
k. Polypropylene
l. Polystyrene
m. Fluoropolymer
n. Any other polymer
o. Any other biocompatible material, or
p. Any other bioresorbable material.

3. A container according to claim 1, wherein the upper part contains pores for gas flow.

4. A container according to claim 1, wherein the upper part contains an upper part central portion which, when inverted, has a raised upper part rim around the upper part central portion.

5. A container according to claim 1, wherein the upper part contains a filter inside.

6. A container according to claim 1, wherein the lower part a central portion has a conic or concave bottom.

7. A container according to claim 6, wherein the lower part central portion has multiple compartments for different biological materials or different experiments.

8. A container according to claim 4, wherein the upper part central portion and/or the lower part central portion contain movable portion that is made of the following biocompatible materials:
a. Glass
b. Chlorotrifluoroethylene—also called ACLAR33C film—
c. Polyvinylchloride
d. Polyethersulfone
e. Polytetrafluoroethylene
f. Polyethylene
g. Polyurethane
h. Polyetherimide
i. Polycarbonate
j. Polysulfone
k. Polyetheretherketone
l. Polypropylene
m. Polystyrene
n. Fluoropolymer
o. Any other polymer
p. Any other biocompatible material
q. Any other bioresorbable material
r. Filtered membrane containing pores with a specific size for the biological material, or
s. Grid for transmission electron microscopy.

9. A container according to claim 1, comprising a cap for the lower part or for, the lower movable central portion that is made of the following materials:
a. Glass
b. Chlorotrifluoroethylene—also called ACLAR33C film—
c. Polyvinylchloride
d. Polyethersulfone
e. Polytetrafluoroethylene
f. Polyethylene
g. Polyurethane
h. Polyetherimide
i. Polycarbonate.
j. Polysulfone
k. Polyetheretherketone
l. Polypropylene
m. Polystyrene
n. Fluoropolymer
o. Any other polymer
p. Any other biocompatible material
q. Any other bioresorbable material
r. Filtered membrane containing pores with a specific size for the biological material, or
s. Grid for transmission electron microscopy.

10. A container according to claim 4, wherein the upper part central portion and/or the lower part central portion are covered and/or filled and/or made off with any of the following reagents:
a. Collagen
b. Matrigel
c. Laminin
b. Fibrinogen
c. Matrigel
d. Hydrogel
e. Alginate
f. Fluoropolymer
g. Any other polymer for transplantation or implantation
h. Synthetic or natural any other extracellular matrix components; or
i. Any other biocompatible materials including various extracellular matrix components.

11. A container according to claim 1, wherein the upper part has an upper part central portion, wherein the upper part central portion and/or the lower part central portion contain scaffolds.

12. A container according to claim 1, comprising multiple niches with different sizes and shapes.

13. A container according to claim 1, wherein the container is designed to provide specific desired shapes and sizes for the culture of the cells needing ultra-low attached surfaces or shaped for adherent surfaces.

14. A container according to claim 1, wherein the lower part and/or upper part have chambers, channels, and layers to provide an environment for co-culture experiments.

15. A container according to claim 1, wherein the holes on the sides of the lower and upper parts provide connection with a microfluidic system.

16. A container according to claim 1, wherein the container is surrounded with a thermal insulator part that is made of the any of the following, but not limited to: Aerogel, fiberglass, cellulose, or polystyrene.
